(19) United States
(12) Patent Application Publication
(10) Pub. No.: US 2025/0270178 A1
(43) Pub. Date: Aug. 28, 2025

(54) METHODS OF PRODUCING A NITRATE ESTER

(71) Applicant: Northrop Grumman Systems Corporation, Falls Church, VA (US)

(72) Inventors: Ryan P. Brisbin, Ogden, UT (US); Christina N. Christensen, Hooper, UT (US)

(21) Appl. No.: 19/066,689

(22) Filed: Feb. 28, 2025

Related U.S. Application Data
(60) Provisional application No. 63/559,119, filed on Feb. 28, 2024.

(51) Int. Cl. C07D 301/00; B01J 31/02; B01J 31/06

(52) U.S. Cl. CPC C07D 301/00; B01J 31/0237; B01J 31/06

(57) ABSTRACT

A method of producing a nitrate ester is disclosed and comprises reacting an alcohol compound and an electrophilic compound to form a sulfonate compound. The sulfonate compound is reacted with a nitrate containing compound to form the nitrate ester. Additional methods of producing glycidyl nitrate or a nitrate ester are also disclosed.

### PRIORITY CLAIM

This application claims the benefit of the filing date of U.S. Provisional Patent Application Ser. No. 63/559,119, filed Feb. 28, 2024, for “METHODS OF PRODUCING NITRATE ESTERS INCLUDING GLYCIDOL NITRATE,” the disclosure of which is hereby incorporated herein in its entirety by this reference.

### TECHNICAL FIELD

Embodiments of the disclosure relate generally to methods of producing a nitrate ester. More particularly, embodiments of the disclosure relate to acid-free methods of producing a nitrate ester.

### BACKGROUND

Conventional methods for forming nitrate esters, such as nitronium-based approaches using mixed acids in batch processes, require a large excess of reagents that are highly corrosive to processing equipment and generate waste streams requiring intensive remediation. While technologies utilizing in-situ generation of dinitrogen pentoxide (N₂O₅) offer potential alternatives, they face challenges in scaling up for industrial production.

Glycidol nitrate (GLYN) is an energetic precursor that is used to produce poly(glycidol nitrate) (PGN), an energetic polymer. The PGN is used as a polymer in a binder system for explosives or propellants. Typically, producing GLYN is a hazardous process that includes two exothermic chemical reactions, uses acids, and generates trinitroglycerol (NG) as a byproduct. Glycerol is nitrated to form a dinitroglycerol (DNG) compound, which is cyclized to form the GLYN. During the nitration reaction, process conditions are closely controlled to prevent runaway reactions. Extensive cooling is also utilized to control the reaction. The thermally unstable oxirane ring of GLYN also contributes to the hazards of the process.

GLYN has been produced by batch or continuous batch processes. The hazards associated with the batch processes are high due to the large volumes and amounts of reagents, intermediates, and reaction products used in large scale production as well as the acidic conditions. The processes also use large volumes of solvent, such as dichloromethane (DCM), that subsequently need to be disposed of. The continuous batch processes have a high capital cost and a reasonable ability to scale up, and there is a high likelihood of hazards due to the large volumes and amounts of reagents, intermediates, and reaction products used for large scale production. Mitigating the hazards in the batch and continuous batch processes has made conventional processes of producing GLYN prohibitively expensive. The large-scale production of GLYN is, therefore, not only dangerous but also expensive.

### BRIEF SUMMARY

A method of producing a nitrate ester is disclosed and comprises reacting an alcohol compound and an electrophilic compound to form a sulfonate compound. The sulfonate compound is reacted with a nitrate containing compound to form the nitrate ester.

A method of producing glycidol nitrate is disclosed and comprises combining a solution comprising glycidol sulfonate and an organic solvent with an aqueous solution comprising a metal nitrate compound and a phase transfer catalyst to form an emulsion. The glycidol sulfonate is reacted with the metal nitrate compound to form glycidol nitrate. The glycidol nitrate is transferred to the organic solvent and metal ions and nitrate ions are transferred to the aqueous solution. The glycidol nitrate is recovered from the organic solvent.

An additional method of producing a nitrate ester is disclosed and comprises reacting a halogenated compound with a nitrate containing compound to form the nitrate ester.

### BRIEF DESCRIPTION OF THE DRAWINGS

For a detailed understanding of the present disclosure, reference should be made to the following detailed description, taken in conjunction with the accompanying drawings, in which like elements have generally been designated with like numerals, and wherein:

FIG. 1 is a process flow diagram of a method of forming a nitrate ester in accordance with embodiments of the disclosure;

FIG. 2 illustrates a phase separation phase of a reaction in accordance with embodiments of the disclosure;

FIG. 3 illustrates a PEG facilitated ion transfer phase followed by mixing to result in a nucleophilic nitration and extraction phase in accordance with embodiments of the disclosure;

FIGS. 4A and 4B are schematic illustrations of systems including one or more microfluidic reactors configured to produce a nitrate ester in accordance with embodiments of the disclosure;

FIGS. 5A-5C are schematic illustrations of systems including one or more Plug Flow Reactors (PFRs) configured to produce a nitrate ester in accordance with embodiments of the disclosure; and

FIG. 6 is a nuclear magnetic resonance (NMR) spectrum of glycidol sulfonate formed according to embodiments of the disclosure.

### DETAILED DESCRIPTION

Methods of producing nitrate esters are disclosed. In some embodiments, the nitrate ester is produced by reacting a nitrate containing compound with a chemical compound (e.g., an alcohol compound). In a first reaction, the alcohol compound is reacted with an electrophilic compound (e.g., a sulfonyl halide) to form a sulfonate compound. The electrophilic compound is formulated to be reactive with the alcohol compound and converts a functional group (e.g., an alcohol group) to a better leaving group. In a second reaction, the sulfonate compound is reacted with the nitrate containing compound to form the nitrate ester. The nitrate containing compound is formulated to be reactive with the sulfonate compound. A catalyst may also be used in the second reaction. The nitrate ester may be produced in a microfluidic reactor or a plug flow reactor (PFR).

In some embodiments, the nitrate ester is produced by reacting a halogenated compound with a nitrate containing compound to form a nitrate ester. The nitrate containing compound is formulated to be reactive with the halogenated compound. In some embodiments, the halogenated compound is reacted with the nitrate containing compound in the presence of an organic catalyst (e.g., a tertiary amine compound). The nitrate ester may be produced in a microfluidic reactor or a PFR.

The reactions to produce the nitrate esters according to embodiments of the disclosure utilize a nucleophilic substitution, where the nitrate containing compound functions as a nucleophile to displace a leaving group from a precursor compound. The precursor compound may be an alcohol compound or a halogenated compound, with the nitrate containing compound formulated to facilitate selective substitution. Producing the nitrate esters by reacting electrophilic compounds and nucleophilic compounds, rather than acids and bases, facilitates nitrate ester formation under milder reaction conditions, reduces the need for corrosive reagents and minimizes byproduct formation. The methods according to embodiments of the disclosure also enhance process safety, improve efficiency, and support more sustainable manufacturing practices.

The nitrate ester may include, but is not limited to, glycidol nitrate, 1,3-dinitropropanol, isosorbide mononitrate, isosorbide dinitrate, ethylene glycol mononitrate, ethylene glycol dinitrate, propylene glycol mononitrate, propylene glycol dinitrate, glyceryl mononitrate, glyceryl dinitrate, butanediol mononitrate, butanediol dinitrate, pentaerythritol tetranitrate, and erythritol tetranitrate.

The nitrate ester produced according to embodiments of the disclosure is in a substantially purified form compared to nitrate esters produced by conventional processes. The nitrate ester is also produced by a relatively safe and quick process compared to conventional batch or continuous processes, and with less expensive and commercially available reagents. Relatively short reaction times may significantly reduce the amount of time needed to produce a desired amount of the nitrate ester. The conversion of an alcohol compound or a halogenated compound to a nitrate ester according to embodiments of the disclosure produces a nitrate ester that is at least comparable in purity and yield to conventional batch or continuous processes of producing nitrate esters. The nitrate ester may also be produced at a higher throughput compared to conventional batch or continuous processes. Unlike conventional processes that use strong acids, such as nitric acid and sulfuric acid, the methods according to embodiments of the disclosure avoid acidic conditions, which minimizes hazardous waste. The methods also produce a stable compound instead of reactive intermediates, allowing for long-term storage and staged reactions. This stability, combined with shorter reaction times, enables efficient scalability for producing the nitrate ester without generating hazardous (e.g., explosive) intermediates.

In some embodiments, a nitrate ester is produced by reacting an alcohol compound with a sulfonyl halide in a first reaction, which converts the alcohol compound into a sulfonate compound with a better leaving group (e.g., a tosyl group, a mesyl group, etc.). In a second reaction, the sulfonate compound is reacted with a nitrate containing compound in the presence of a phase transfer catalyst (PTC) to produce the nitrate ester.

In some embodiments, a nitrate ester is produced by reacting a halogenated compound with a nitrate containing compound to produce the nitrate ester. The nitrate containing compound displaces a leaving group (e.g., the halogen) of the halogenated compound to produce the nitrate ester. In some embodiments, the halogenated compound is reacted with the nitrate containing compound in the presence of an optional organic catalyst (e.g., a tertiary amine compound). The organic catalyst displaces a leaving group (e.g., the halogen) of the halogenated compound to form an activated complex with a better leaving group. The activated complex reacts with the nitrate containing compound to form the nitrate ester.

Glycidol nitrate may also be known as glycidyl nitrate. In some embodiments, the method comprises reacting glycidol and a tosyl halide or a mesyl halide to produce glycidol tosylate or glycidol mesylate. A nitrating reagent (NR) is reacted with the glycidol tosylate or glycidol mesylate to form the glycidol nitrate (GLYN). The nitrating reagent includes a metal nitrate compound, which is used with a phase transfer catalyst and water in a nitrating solution. The aqueous nitrating solution is reacted with a solution of the glycidol tosylate or glycidol mesylate to form the glycidol nitrate. The glycidol tosylate or glycidol mesylate is reacted with the aqueous nitrating solution in, for example, a microfluidic reactor (e.g., an Advanced Flow Reactor (AFR)) or a plug flow reactor (PFR) to produce the GLYN. A dinitroglycerol byproduct may optionally be reacted with a base by an intramolecular condensation reaction in the microfluidic reactor to form additional glycidol nitrate. The nitration reaction, the optional intramolecular condensation reaction, and separations are conducted in a system that includes the microfluidic reactor, enabling the GLYN to be produced substantially continuously. Since the reactions and the separations may be conducted in a single system, the GLYN is produced continuously.

As used herein, the singular forms following “a,” “an,” and “the” are intended to include the plural forms as well, unless the context clearly indicates otherwise.

As used herein, the term “about” or “approximately” in reference to a numerical value for a particular parameter is inclusive of the numerical value and a degree of variance from the numerical value that one of ordinary skill in the art would understand is within acceptable tolerances for the particular parameter. For example, “about” or “approximately” in reference to a numerical value may include additional numerical values within a range of from 90.0 percent to 110.0 percent of the numerical value, such as within a range of from 95.0 percent to 105.0 percent of the numerical value, within a range of from 97.5 percent to 102.5 percent of the numerical value, within a range of from 99.0 percent to 101.0 percent of the numerical value, within a range of from 99.5 percent to 100.5 percent of the numerical value, or within a range of from 99.9 percent to 100.1 percent of the numerical value.

As used herein, the term “and/or” includes any and all combinations of one or more of the associated listed items.

As used herein, spatially relative terms, such as “beneath,” “below,” “lower,” “bottom,” “above,” “upper,” “top,” “front,” “rear,” “left,” “right,” and the like, may be used for ease of description to describe one element's or feature's relationship to another element(s) or feature(s) as illustrated in the figures. Unless otherwise specified, the spatially relative terms are intended to encompass different orientations of the materials in addition to the orientation depicted in the figures. For example, if materials in the figures are inverted, elements described as “below” or “beneath” or “under” or “on bottom of” other elements or features would then be oriented “above” or “on top of” the other elements or features. Thus, the term “below” can encompass both an orientation of above and below, depending on the context in which the term is used, which will be evident to one of ordinary skill in the art. The materials may be otherwise oriented (e.g., rotated 90 degrees, inverted, flipped, etc.) and the spatially relative descriptors used herein interpreted accordingly.

As used herein, the terms “comprising,” “including,” “containing,” “characterized by,” and grammatical equivalents thereof are inclusive or open-ended terms that do not exclude additional, unrecited elements or method steps, but also include the more restrictive terms “consisting of” and “consisting essentially of” and grammatical equivalents thereof.

As used herein, the term “configured” refers to a size, shape, material composition, and arrangement of one or more of at least one structure and at least one apparatus facilitating operation of one or more of the structures and the apparatus in a pre-determined way. As used herein, the term “about” used in reference to a given parameter is inclusive of the stated value and has the meaning dictated by the context (e.g., it includes the degree of error associated with measurement of the given parameter, as well as variations resulting from manufacturing tolerances, etc.).

As used herein, the term “may” with respect to a material, structure, feature or method act indicates that such is contemplated for use in implementation of an embodiment of the disclosure and such term is used in preference to the more restrictive term “is” so as to avoid any implication that other, compatible materials, structures, features and methods usable in combination therewith should or must be excluded.

As used herein, the term “microfluidic reactor” means and includes a vessel (e.g., a reactor) configured to conduct chemical reactions under geometrically constrained conditions. The reactor includes a reaction channel having internal dimensions on the μm scale, such as between about 1 μm and about 1000 μm, and a reaction volume of less than or equal to about 40 ml. For example, the reaction channel may have an inner diameter of less than or equal to about 1000 μm.

As used herein, the term “nitrating reagent” means and includes a nitrate containing compound that is formulated to be reactive with a chemical compound containing a leaving group, such as, but not limited to, a sulfonyl halide functional group or a halogen functional group. The nitrating reagent provides a source of nitrate ions for reacting with the chemical compound. The nitrate anions function as a nucleophile and react with an electrophilic atom of the chemical compound. The nitrating reagent may be formulated in a nitrating reagent solution that also includes a phase transfer catalyst and water. By way of example only, the chemical compound may be a glycidol tosylate or a glycidol mesylate. Alternatively, the nitrating reagent may be formulated in a nitrating reagent solution including an organic catalyst. By way of example only, the chemical compound may be a haloalcohol.

As used herein, the term “reaction volume” means and includes a volume of a reaction channel of the microfluidic reactor within which the reaction(s) is conducted.

As used herein, the term “residence time” means and includes a total time the reaction solution including the reagents is in the microfluidic reactor. The residence time is a function of the reaction volume of the microfluidic reactor and of a flow rate that the reaction solution is flowed through the microfluidic reactor. The residence time corresponds to the amount of time for the reaction volume to move through the microfluidic reactor.

As used herein, the term “substantially,” in reference to a given parameter, property, or condition, means to a degree that one of ordinary skill in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

The following description provides specific details, such as reagents, reagent amounts, and reaction conditions in order to provide a thorough description of embodiments of the disclosure. However, a person of ordinary skill in the art will understand that the embodiments of the disclosure may be practiced without employing these specific details. Indeed, the embodiments of the disclosure may be practiced in conjunction with conventional processes employed in the industry. In addition, the description provided below does not form a complete process flow for producing the nitrate esters. Only those process acts necessary to understand the embodiments of the disclosure are described in detail below. Additional acts may be performed by conventional techniques. Also note, any drawings accompanying the application are for illustrative purposes only and are not drawn to scale. Additionally, elements common between figures may retain the same numerical designation.

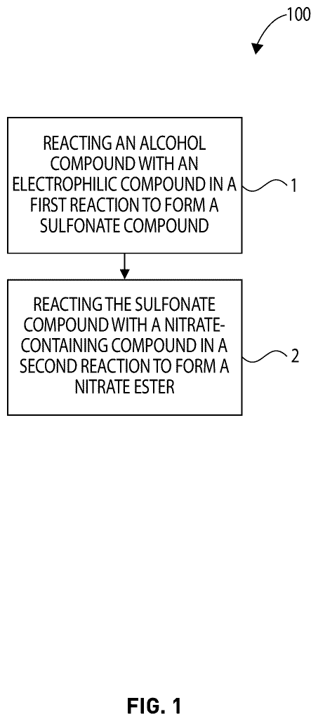

FIG. 1 illustrates a process flow 100 showing the production of a nitrate ester from an alcohol compound. The process flow 100 includes act 1 of reacting an alcohol compound with an electrophilic compound in a first reaction to form a sulfonate compound and act 2 of reacting the sulfonate compound with a nitrate containing compound in a second reaction to form the nitrate ester. The nitrate ester may be produced by an acid-free nitration method. In other words, no acid is used during the nitration reaction. The production of nitrate esters from alcohol compounds may be conducted in a microfluidic reactor or a larger volume reactor, such as a PFR.

The nitrate ester is produced by a liquid-liquid phase transfer mechanism that proceeds by a biphasic reaction that includes an organic phase and an aqueous phase according to the following general reaction scheme (where “SG” refers to a sulfonate group and R is an unsaturated, branched, straight-chain, cyclic, or other alkyl group including from 3 to 10 carbon atoms):

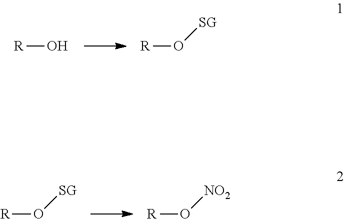

According to some embodiments of the disclosure, the alcohol compound is converted to a sulfonate compound in a first reaction. In the first reaction of act 1, a functional group (e.g., an alcohol group) is converted to a better leaving group (e.g., a sulfonate group) by reaction with an electrophilic compound (e.g., a tosyl- or mesyl-halide, etc.). The sulfonate compound is reacted with the nitrate containing compound (i.e., the nitrating reagent (NR)) and converted to the nitrate ester in the second reaction of act 2, which is a nucleophilic substitution reaction, such as by an S_{N}1/S_{N}2 reaction. The loss of the leaving group is followed by or concurrent with reaction of a nucleophile (e.g., the nitrate ions) with the carbocation. The alcohol compound and electrophilic compound are commercially available from numerous sources. Other reagents used in the first and second reactions are also commercially available, which enables the nitrate ester to be produced from less expensive starting materials and reagents than conventional techniques of forming nitrate esters.

Act 1 of reacting an alcohol compound with an electrophilic compound in a first reaction to form a sulfonate compound may include reacting the alcohol compound (e.g., glycidol) with the electrophilic compound (e.g., a sulfonyl halide, such as tosyl- or mesyl-halide) in the presence of a base and a solvent at a controlled temperature, such as below about 25° C., to form a sulfonate compound (e.g., glycidol tosylate (GT) or glycidol mesylate). While specific examples herein describe using tosyl chloride in the first reaction, other tosyl- or mesyl-halides may be used, such as mesyl chloride, tosyl iodide, or mesyl iodide. Furthermore, while specific examples herein disclose using glycidol in the first reaction, other alcohol compounds may be used. The base used in the first reaction may be an amine or heterocyclic base, such as one or more of triethylamine (TEA), pyridine, lutidine, dimethylamino pyridine, etc. The solvent may be DCM, acetonitrile (ACN), tetrahydrofuran (THF), acetone, other polar aprotic solvent, or other halogenated or non-protic solvent. The alcohol compound, the electrophilic compound, the base, and the solvent may, for example, be reacted in solution to form the sulfonate compound.

The first reaction may be performed at a low temperature, such as at a temperature below room temperature (below about 25° C.) since the first reaction is exothermic. At a temperature above room temperature, the sulfonate compound yield may decrease and regioselectivity may be reduced. The first reaction may, for example, be conducted at a temperature of about 0° C. The first reaction is conducted at a reduced temperature and produces the sulfonate compound in the form of a solid precipitate. The first reaction may be conducted as a batch process or as a continuous process. In some embodiments, the sulfonate compound is produced in a microreactor. The sulfonate compound in the sulfonate compound solution may be purified to from about 95% to about 99% by recrystallization or other conventional techniques prior to conducting the second reaction. The sulfonate compound may be formed in a batch process or a continuous process, such as a flow process using a microfluidic reactor.

In some embodiments, the sulfonate compound is formed according to the following reaction scheme when the alcohol compound is glycidol and the sulfonate group is a tosyl group:

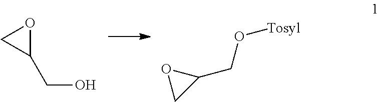

Act 2 of reacting the sulfonate compound with a nitrate containing compound in the presence of a PTC in a second reaction to form a nitrate ester may include dissolving the sulfonate compound (i.e., the glycidol tosylate) in an organic solvent, such as acetonitrile, to produce a sulfonate compound solution. However, other solvents may be used to dissolve the sulfonate compound with a nitrate containing compound in the presence of a PTC in a second reaction to form a nitrate ester.

During the second reaction, the sulfonate compound in the sulfonate compound solution is converted to a nitrate ester, e.g., glycidol nitrate (GLYN), by reacting the sulfonate compound with the nitrating reagent in a NR solution. The nitrating reagent may be a metal nitrate compound. The metal nitrate compound may be a cationic, metal nitrate compound, including, but not limited to, sodium nitrate, potassium nitrate, calcium nitrate, cesium nitrate, copper nitrate, manganese nitrate, or rubidium nitrate. The NR solution includes the nitrating reagent, which functions as a nucleophile, the phase transfer catalyst and a solvent. A surfactant may, optionally, be present in the NR solution. The nitrating reagent, such as sodium nitrate, may be dissolved in water (e.g., distilled water) and mixed with the phase transfer catalyst, the optional surfactant, and the solvent to form the NR solution.

An excess of the nitrating reagent may be used in the second reaction to produce the nitrate ester. In some embodiments, the nitrating reagent is sodium nitrate and the NR solution is an aqueous solution. The sodium nitrate is present at from about 0.5 M sodium nitrate to about 10.0 M sodium nitrate, such as from about 1.0 M sodium nitrate to about 10 M sodium nitrate, from about 1.5 M sodium nitrate to about 8.0 M sodium nitrate, from about 2.0 M sodium nitrate to about 7.0 M sodium nitrate, from about 2.0 M sodium nitrate to about 6.0 M sodium nitrate, from about 3.0 M sodium nitrate to about 6.0 M sodium nitrate, or from about 3.5 M sodium nitrate to about 5.5 M sodium nitrate. Similar concentrations may be used if the nitrating reagent is a different metal nitrate compound. By using a concentrated aqueous solution of the nitrating reagent to combine with the sulfonate compound solution, the second reaction may be conducted as a liquid-liquid phase reaction. The high concentration of the nitrating reagent used may also decrease the reactivity of water during the nitration reaction, as measured by minimal or no hydrolysis products being produced. The NR solution including the nitrating reagent is also safer to work with than nitric acid or a combination of nitric acid and sulfuric acid, which are used in conventional techniques.

The phase transfer catalyst may be a linear polyether, a cyclic polyether, a substituted linear polyether, a substituted cyclic polyether, an isotopic isomer of the linear polyether, an isotopic isomer of the cyclic polyether, or a combination thereof. By way of example only, the phase transfer catalyst may be polyethylene glycol (PEG), 18-crown-6, hexadecyl pyridinium bromide, a surfactant, EDTA, or a combination thereof. The phase transfer catalyst may exhibit a molecular weight within a range of from about 150 to about 10,000. If, for example, the phase transfer catalyst is PEG, the PEG may exhibit an average molecular weight of from about 1000 to about 10,000. The phase transfer catalyst may be commercially available from numerous sources. In some embodiments, the phase transfer catalyst is PEG. Presolvated PEG may be used, which has been found to increase kinetics and efficacy of the second reaction. The phase transfer catalyst may present in the NR solution at a concentration of from about 0.001 M to about 0.100 M, such as from about 0.001 M to about 0.008 M, from about 0.001 M to about 0.006 M, from about 0.002 M to about 0.006 M, or from about 0.003 M to about 0.006 M. The NR solution may include a catalytic amount of the phase transfer catalyst, such as from about 0.01 mol % to about 15 mol %, from about 2 mol % to about 15 mol %, from about 5 mol % to about 15 mol %, from about 8 mol % to about 15 mol %, from about 8 mol % to about 12 mol %, or from about 9 mol % to about 11 mol %.

The surfactant used in the NR solution may be a cationic surfactant, such as a quaternary ammonium salt or hexadecyl pyridinium bromide. In some embodiments, anionic surfactants or chelators, such as EDTA, may be used. The surfactant in the NR solution may be present at a concentration of from about 0.001 M to about 0.100 M, such as from about 0.001 M to about 0.008 M, from about 0.001 M to about 0.006 M, from about 0.002 M to about 0.006 M, or from about 0.003 M to about 0.006 M. If a surfactant is present in the NR solution, the surfactant may be present in addition to the phase transfer catalyst.

The nitrating reagent, PTC, and optional surfactant may be combined in water to form the NR solution. Alternatively, the PTC may be combined with the sulfonate compound solution rather than the NR solution.

The sulfonate compound solution and the NR solution are combined with mixing, following which the sulfonate compound is nitrated by the nitrating reagent. A ratio of the NR solution to the sulfonate compound solution may range from about 1:1 to about 10:1, such as from about 1:1 to about 5:1, from about 5:1 to about 10:1, or from about 4:1 to about 8:1. The sulfonate compound solution and the NR solution may be introduced into a reactor and combined. By way of example only, the sulfonate compound solution and the NR solution may be pumped into a microfluidic reactor.

In some embodiments, the nitrate ester (e.g., GLYN) is formed according to the following reaction scheme:

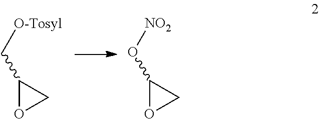

The second reaction may take place at a temperature ranging from about 60° C. to about 200° C., such as from about 75° C. to about 185° C., from about 90° C. to about 170° C., from about 105° C. to about 165° C., from about 120° C. to about 150° C., from about 150° C. to about 200° C., from about 160° C. to about 195° C., from about 170° C. to about 190° C., or from about 175° C. to about 185° C. In some embodiments, the nitration reaction is conducted at a reaction temperature of about 195° C. In other embodiments, the nitration reaction is conducted at a reaction temperature of about 160° C. In yet other embodiments, the nitration reaction is conducted at a reaction temperature of about 175° C.

Without being bound by any theory, it is believed that the second reaction (e.g., the nitration reaction) may occur in distinct and separate phases that include, but are not limited to, an inert phase separation, an emulsion formation phase, a PTC facilitated ion transfer phase, and a nucleophilic nitration and extraction phase. During the nitration reaction, liberation of an anionic species from its solid-state crystal lattice (solvating of nitrate from solid sodium nitrate) occurs. Without the presence of a PTC, the liberation process is slow from a kinetic standpoint, largely relying on intercalation mechanisms and literal morphological changes at the crystal surface. However, with the PTC, an increase in solubility of the nitrating reagent is observed.

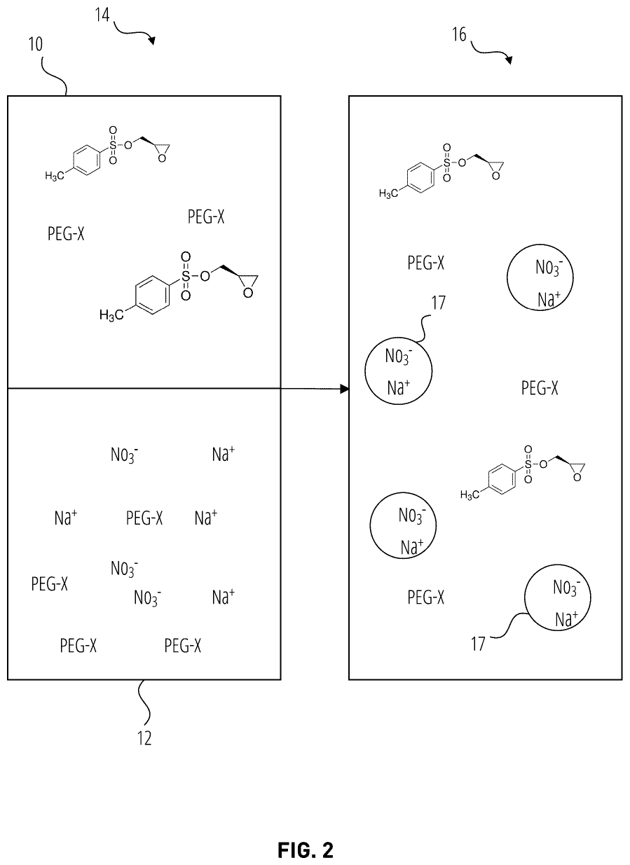

The inert phase separation may occur at the beginning of the second reaction, during which there is limited to no reactivity between the sulfonate compound and the NR because the reagents are separated into organic and aqueous phases (e.g., are phase separated). While water of the NR solution and the solvent (e.g., acetonitrile) of the sulfonate compound solution would be expected to be miscible, the nitrating reagent is insoluble in the solvent, which leads to a biphasic system that includes water and an organic solvent. As shown in FIG. 2, an organic phase 10 separates from an aqueous phase 12 during an inert phase separation 14. The sodium nitrate, which is insoluble in the organic phase 10 (e.g., acetonitrile), forces the phase separation into the organic and aqueous phases 10, 12. The high concentration of the nitrating reagent in the aqueous phase 12 reduces the nucleophilicity of water, reducing or substantially eliminating the reactivity of the water. The nitrating reagent decreases the reactivity of water during the nitration reaction, as measured by minimal or no hydrolysis products being produced. As the second reaction progresses, the sodium nitrate moves to the organic phase 10, forming nitrate ions which function as a nucleophile. The PTC (e.g., PEG) is soluble in both the organic and aqueous phases 10, 12. The PTC (e.g., PEG) may be presolvated for increasing kinetics and efficacy of the second reaction. The GT and PEG are, therefore, initially present in the organic phase 10 and ions (e.g., Na⁺, NO₃⁻) and PEG are initially present in the aqueous phase 12.

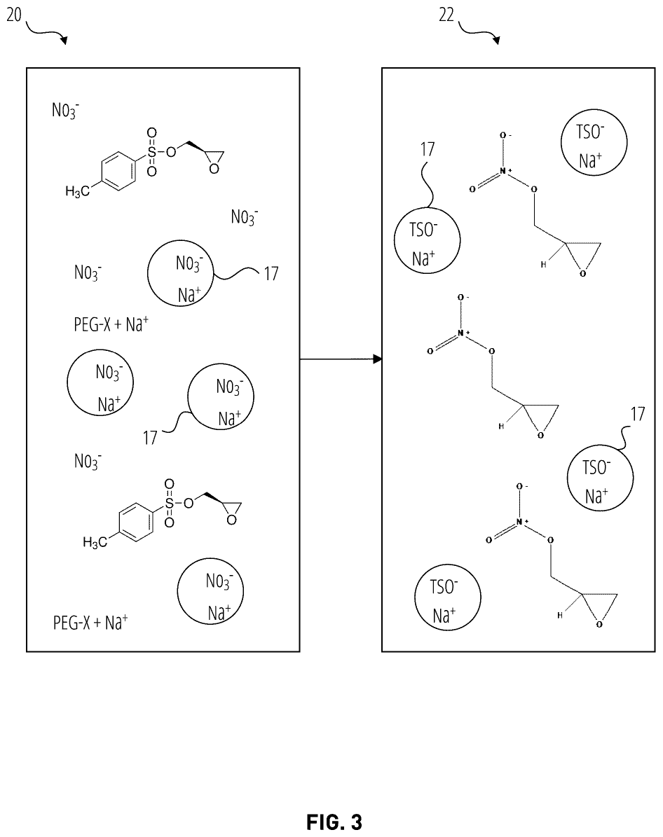

With continued reference to FIG. 2, the organic and aqueous phases 10, 12 are mixed to form an emulsion formation phase 16, which gives rise to water microdroplets 17 on the immiscible water and organic layers. The emulsion formation phase 16 includes the GT, PEG, Na⁺ and NO₃⁻. During a PTC facilitated ion transfer phase 20, as shown in FIG. 3, the PTC (e.g., PEG) chelates the sodium ions (Na⁺) allowing for the solubilizing of nitrate ions (NO₃⁻). A nucleophilic nitration and extraction phase 22 then occurs where the solubilized nitrate ions react with the electrophile, displacing the tosylate anion, and the water microdroplets 17 remove the tosylate anion from the organic layer. The NO₃⁻ anions react with the carbon atom alpha to the oxygen atom on the GT, displacing the tosylate and forming GLYN. Since the aqueous phase following the nitration reaction has a neutral pH, the aqueous phase may be easily disposed of.

The nitration reaction takes place in the microfluidic reactor to form GLYN in the organic phase and an aqueous phase having a neutral pH (e.g., a pH of about 7.0). Using the microfluidic reactor may enable sufficient heat transfer and mixing for the nitration reaction to occur. Mixing within the microfluidic channels of the microfluidic reactor facilitates the formation of the emulsion. A high surface area to volume ratio within the microfluidic reactor provides a consistent temperature to be maintained.

The reactants of the second reaction may be introduced into a first reaction channel of the microfluidic reactor at a flow rate sufficient for the reaction to take place to substantial completion in the reaction channel. The flow rate may be selected to achieve the desired NR: GT ratio and residence time. The flow rate may, for example, be within a range of from about 0.05 ml/minute to about 10.00 ml/minute, from about 0.05 ml/minute to about 8.00 ml/minute, from about 0.05 ml/minute to about 7.50 ml/minute, from about 0.05 ml/minute to about 7.00 ml/minute, from about 0.10 ml/minute to about 6.00 ml/minute, from about 0.10 ml/minute to about 5.50 ml/minute, from about 0.10 ml/minute to about 5.00 ml/minute, from about 0.10 ml/minute to about 4.50 ml/minute, from about 0.10 ml/minute to about 4.00 ml/minute, from about 0.10 ml/minute to about 3.50 ml/minute, from about 0.10 ml/minute to about 3.00 ml/minute, from about 0.10 ml/minute to about 2.50 ml/minute, from about 0.10 ml/minute to about 2.00 ml/minute, from about 0.10 ml/minute to about 1.50 ml/minute, from about 0.10 ml/minute to about 1.25 ml/minute, from about 0.125 ml/minute to about 1.25 ml/minute, from about 0.15 ml/minute to about 1.25 ml/minute, from about 0.25 ml/minute to about 1.25 ml/minute, or from about 0.75 ml/minute to about 1.25 ml/minute. In some embodiments, the flow rate is 3.00 ml/minute. In other embodiments, the flow rate is 1.10 ml/minute. In yet other embodiments, the flow rate is 0.44 ml/minute. The flow rate of the NR and the GT solutions may be the same as one another or may be different.

The residence time of the reaction products of the nitration reaction may range from about 0.25 minutes to about 5.0 minutes, such as from about 0.25 minutes to about 3.0 minutes, from about 0.5 minutes to about 2.0 minutes, from about 0.75 minutes to about 1.75 minutes, or from about 1.0 minutes to about 1.5 minutes. In some embodiments, the residence time is about 0.3 minutes. In other embodiments, the residence time is about 1.25 minutes. The short reaction time for conducting the nitration reaction was unexpected relative to the reaction times of conventional nitration processes.

A ratio of the nitrating reagent to glycidol tosylate (NR:GT) may range from about 1.0:1.0 to about 10.0:1.0, such as from about 2.0:1.0 to about 10.0:1.0, 3.0:1.0 to about 10.0:1, from about 4.0:1.0 to about 10.0:1.0, from about 4.0:1.0 to about 8.0:1.0, from about 3.0:1.0 to about 8.0:1.0, from about 3.0:1.0 to about 6.0:1.0, from about 3.0:1.0 to about 5.0:1.0, or from about 3.5:1.0 to about 4.5:1.0. In some embodiments, the ratio of nitrating reagent to glycidol tosylate is about 4.0:1.0. In other embodiments, the ratio of nitrating reagent to glycidol tosylate is about 8.0:1.0. The NR: GT ratio corresponds to a ratio of flow rates from pumps used to deliver each of the NR solution and the GT solution to the reactor.

While embodiments describe the second (e.g., nitration) reaction as being conducted in the microfluidic reactor, both the first and second reactions may be conducted in the microfluidic reactor. Therefore, the GLYN may be produced in a single, enclosed system. The GLYN may be isolated for collection from a non-flammable solvent, such as dichloromethane.

By forming the GLYN without using an acid reagent, acidic reaction conditions and the disposal of large volumes and concentrations of acid are avoided. In contrast, conventional techniques of forming GLYN use nitric acid or a combination of nitric acid and sulfuric acid. Therefore, the nitration reaction may be conducted at a neutral pH, in contrast to the acidic conditions that are used with conventional techniques of forming GLYN. In addition, less expensive starting materials and reagents are used since the starting materials are commercially available compared to those used in conventional techniques of forming GLYN. The hazards associated with conventional techniques of forming GLYN may also be mitigated since the reagents are safer and do not produce high volumes of hazardous waste streams. The product of the first reaction is a stable compound compared to reactive, intermediate compounds produced during conventional techniques of forming GLYN. The product of the first reaction may, therefore, be stored for long periods of time. The stability also enables the first and second reactions to be conducted at discrete times, with storage of the products possible. Thus, the GLYN may be produced by a so-called “green” and sustainable process. The formation of GLYN according to embodiments of the disclosure may also be easily scaled up to produce large quantities without producing hazardous (e.g., energetic, explosive) intermediate compounds.

In addition to being efficient, the GLYN formed by the methods according to embodiments of the disclosure was, unexpectedly, found to not produce hydrolysis products. With conventional processes of producing GLYN, water is known to decompose nitramines and nitrate esters. Conducting the nitration using an aqueous system was, therefore, expected to decompose nitramines and nitrate esters. However, it was unexpectedly determined that the aqueous system of the second reaction did not hydrolyze or decompose the resulting GLYN. The resulting GLYN may be produced at a molar purity of greater than about 90%, such as greater than about 95% or greater than about 95% as measured (e.g., analyzed) by conventional NMR or HPLC techniques.

While embodiments describe producing GLYN, similar methods may be used to produce other nitrate ester compounds. For example, the nitrate ester compound may be produced by reacting an alcohol compound other than glycidol with a sulfonyl halide to produce a sulfonate compound. A solution comprising the sulfonate compound and an organic solvent may be formed and an aqueous solution comprising a nitrate compound and a phase transfer catalyst may be formed. The nitrate compound in the aqueous solution may be reacted with the sulfonate compound in the organic phase to produce the nitrate ester compound. Alternatively, the nitrate ester compound may be produced by reacting the halogenated compound with an organic catalyst (e.g., a tertiary amine) to produce a quaternary ammonium intermediate. The quaternary ammonium intermediate may be reacted with a nitrate compound to produce the nitrate ester compound. The resulting nitrate ester compound may include, but is not limited to, 1,3-dinitropropanol, isosorbide mononitrate, isosorbide dinitrate, ethylene glycol mononitrate, ethylene glycol dinitrate, propylene glycol mononitrate, propylene glycol dinitrate, glyceryl mononitrate, glyceryl dinitrate, butanediol mononitrate, butanediol dinitrate, pentaerythritol tetranitrate, and erythritol tetranitrate. The alcohol compound may include, but is not limited to, glycidol, isosorbide, ethylene glycol, propylene glycol, glycerol, 1,4-butanediol, pentaerythritol, or erythritol, isosorbide, ethylene glycol, propylene glycol, glycerol, 1,4-butanediol, pentaerythritol, or erythritol.

While embodiments describe producing GLYN in a microfluidic reactor, a larger volume reactor, such as a meso-scale reactor, may be used to form the GLYN or other nitrate ester compound. The methods according to embodiments of the disclosure may be scaled up and conducted in a larger volume reactor depending on a desired amount of GLYN to be produced. The GLYN may be produced by a continuous process or by a batch process.

Other nitrated glycerol compounds, such as a mononitroglycerol (MNG) compound or a dinitroglycerol (DNG) compound may be formed as byproducts during the nitration reaction. The DNG may, for example, be 1,3-DNG. The MNG or DNG byproducts may be formed due to the nucleophile/electrophile reaction mechanism. To achieve an increased yield of GLYN, the DNG compound may optionally be converted to GLYN by an intramolecular ring closure reaction. The intramolecular ring closure reaction may be conducted in a second channel of the microfluidic reactor coupled to the channel in which the nitration reaction occurs. The amount of DNG compound produced during the nitration reaction may be maximized so that GLYN and the DNG compound are major products, with trace amounts or substantially no MNG compound or NG produced. The DNG compound may, therefore, be a major byproduct of the nitration reaction compared to the relative amount of MNG compound or NG produced. The MNG compound, if present, reduces the purity of the resulting GLYN and affects subsequent polymerization of the GLYN. By substantially reducing or eliminating the NG in methods according to embodiments of the disclosure, hazards associated with conventional nitration reactions are reduced or eliminated. Reaction temperature, starting materials, and residence time may be adjusted to produce the GLYN as the major reaction product and DNG as a major byproduct of the nitration reaction.

The DNG compound may be reacted with a base in the second microfluidic reactor to induce an intramolecular ring closure and form GLYN. The base may include, but is not limited to, sodium hydroxide (NaOH), and a concentration of the sodium hydroxide may range from about 0.1 M to about 10.0 M, such as from about 5.0 M to about 8.0 M, from about 6.0 M to about 9.0 M, or from about 7.0 M to about 8.0 M. The sodium hydroxide may be an aqueous solution of sodium hydroxide. In some embodiments, the concentration of sodium hydroxide in the aqueous solution of sodium hydroxide is about 5.5 M.

A volume ratio of the DNG compound to the base may range from about 2.0:1 to about 4.5:1, such as from about 2.5:1 to about 4.0:1, from about 2.5:1 to about 3.5:1, or from about 2.5:1 to about 3.0:1. In some embodiments, the ratio of the DNG compound to the base is 2.7:1.

The DNG compound and the base may be reacted in the microfluidic reactor at a temperature of from about 10° C. to about 120° C., such as from about 10° C. to about 20° C., from about 10° C. to about 15° C., from about 15° C. to about 25° C., from about 15° C. to about 20° C., from about 20° C. to about 25° C., from about 20° C. to about 35° C., from about 25° C. to about 35° C., or from about 30° C. to about 35° C. In some embodiments, the temperature is about 14° C. In other embodiments, the temperature is about 20° C. In yet other embodiments, the temperature is about 22° C. In yet other embodiments, the temperature is about 32° C. Controlling the temperature of the intramolecular condensation reaction may produce the GLYN at a higher purity by decreasing side reactions.

The DNG and the base may be introduced into a second microfluidic reactor at a flow rate within a range of from about 0.05 ml/minute to about 10.00 ml/minute, from about 0.10 ml/minute to about 9.00 ml/minute, from about 0.50 ml/minute to about 8.00 ml/minute, from about 0.50 ml/minute to about 7.00 ml/minute, from about 0.50 ml/minute to about 6.50 ml/minute, from about 0.50 ml/minute to about 6.00 ml/minute, from about 0.50 ml/minute to about 5.50 ml/minute, from about 0.50 ml/minute to about 5.00 ml/minute, from about 0.05 ml/minute to about 2.50 ml/minute, from about 0.10 ml/minute to about 1.25 ml/minute, from about 0.125 ml/minute to about 1.25 ml/minute, from about 0.15 ml/minute to about 1.25 ml/minute, from about 0.25 ml/minute to about 1.0 ml/minute, from about 0.50 ml/minute to about 1.0 ml/minute, or from about 0.75 ml/minute to about 1.0 ml/minute. In some embodiments, the flow rate is between about 0.50 ml/minute and about 1.0 ml/minute. In other embodiments, the flow rate is between about 0.50 ml/minute and about 2.5 ml/minute.

The residence time of the intramolecular condensation reaction may range from about 0.5 minutes to about 30.0 minutes, such as from about 2.0 minutes to about 25.0 minutes, from about 2.0 minutes to about 25.0 minutes, from about 5.0 minutes to about 25.0 minutes, from about 10.0 minutes to about 25.0 minutes, from about 15.0 minutes to about 25.0 minutes, from about 20.0 minutes to about 25.0 minutes, from about 1 minute to about 15 minutes, from about 2 minutes to about 10 minutes, from about 4 minutes to about 8 minutes, or from about 4 minutes to about 6 minutes. In some embodiments, the residence time is about 1.2 minutes. In other embodiments, the residence time is about 0.7 minutes. In comparison, converting the DGN compound to GLYN by conventional batch processes takes between 2 hours and 3 hours.

By using the optional intramolecular condensation reaction along with the nitration reaction, an increased amount of GLYN may be produced relative to that produced by the nitration reaction alone. By way of example only, the nitration reaction may produce GLYN at from about 45 mol % to about 90 mol %, such as from about 45 mol % to about 66 mol % as determined by HPLC or NMR analysis. Following conversion of DNG to GLYN using the optional intramolecular condensation reaction, up to about 95 mol % of the GLYN was produced. The final purity was determined to be from about 85 mol % to about 95 mol % GLYN as determined by NMR analysis.

The GLYN produced by embodiments of the disclosure may be used as a precursor to produce poly(glycidol nitrate) (PGN), an energetic polymer. The PGN may, for example, be used in explosive compositions or propellant compositions. The GLYN may also be used as a precursor for pharmaceutical use.

In some embodiments, a nitrate ester is produced in a monophasic reaction without the use of a PTC. By way of example only, an organic catalyst may be used to produce the nitrate ester without the use of a PTC. For example, a halogenated compound may be reacted with a nitrate containing compound to form the nitrate ester. Alternatively, a halogenated compound may be reacted with the organic catalyst in a first reaction to form an activated complex of the halogenated compound. The activated complex of the halogenated compound may be reacted with a nitrate containing compound in a second reaction to form the nitrate ester.

The nitrate ester may be produced in the monophasic reaction by reacting the halogenated compound with the nitrate containing compound to form the nitrate ester. The nitrate containing compound may displace a leaving group (e.g., the halogen) of the halogenated compound to form the nitrate ester. For example, 1,3-dichloropropanol may be reacted with a nitrate containing compound (e.g., sodium nitrate) to form 1,3-dinitropropanol. The reaction may be performed at a temperature within a range of from about 60° C. to about 200° C., such as from about 75° C. to about 185° C., from about 90° C. to about 170° C., from about 105° C. to about 165° C., from about 120° C. to about 150° C., from about 150° C. to about 200° C., from about 160° C. to about 195° C., from about 170° C. to about 190° C., or from about 175° C. to about 185° C.

In embodiments where the nitrate ester is produced using an organic catalyst, the nitrate ester is produced in a monophasic reaction according to the following general reaction scheme (where “LG” refers to leaving group):

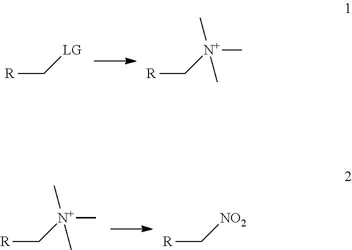

The organic catalyst may include a tertiary amine, such as, but not limited to, 1,4-diazabicyclo[2.2.2]octane (DABCO), triethylamine (TEA), N,N-dimethylaminopyridine (DMAP), 1,8-diazabicyclo[5.4.0]undec-7-ene (DBU), N-methylmorpholine (NMM), 1,4-diazabicyclo[2.2.1]heptane (DABCH), N,N-diisopropylethylamine (DIPEA), quinuclidine, N-methylpiperidine, N,N,N′,N′-tetramethylethylenediamine (TMEDA), 1,5-diazabicyclo[4.3.0]non-5-ene (DBN), 2,2,6,6-tetramethylpiperidine (TMP), or combinations thereof. The organic catalyst may react with the halogenated compound to displace the leaving group (e.g., the halogen, such as, but not limited to, chloride, fluoride, bromide, or iodide) from the halogenated compound and form an activated complex in the form of a quaternary ammonium cation. For example, a halogenated compound such as 1,3-dichloropropanol (1,3-DCP) may be reacted with the tertiary amine catalyst to form a quaternary ammonium intermediate. This intermediate has increased electrophilicity while simultaneously providing a good leaving group in the form of a charged nitrogen species.

The quaternary ammonium intermediate may then undergo nucleophilic substitution with nitrate anions of the nitrate containing compound to form the nitrate ester (e.g., 1,3-dinitropropanol (1,3-DNP)). This nitration reaction with the organic catalyst (e.g., without the use of the PTC) may be performed at a temperature within a range of from about 60° C. to about 200° C., such as from about 75° C. to about 185° C., from about 90° C. to about 170° C., from about 105° C. to about 165° C., from about 120° C. to about 150° C., from about 150° C. to about 200° C., from about 160° C. to about 195° C., from about 170° C. to about 190° C., or from about 175° C. to about 185° C.

The nitrate ester formed in a monophasic reaction without the use of a PTC may be produced in a microfluidic reactor under substantially similar conditions (e.g., flow rates, residence times, and ratios of reagents) as described above in relation to FIG. 2 and FIG. 3, or in a plug flow reactor, as described in further detail below.

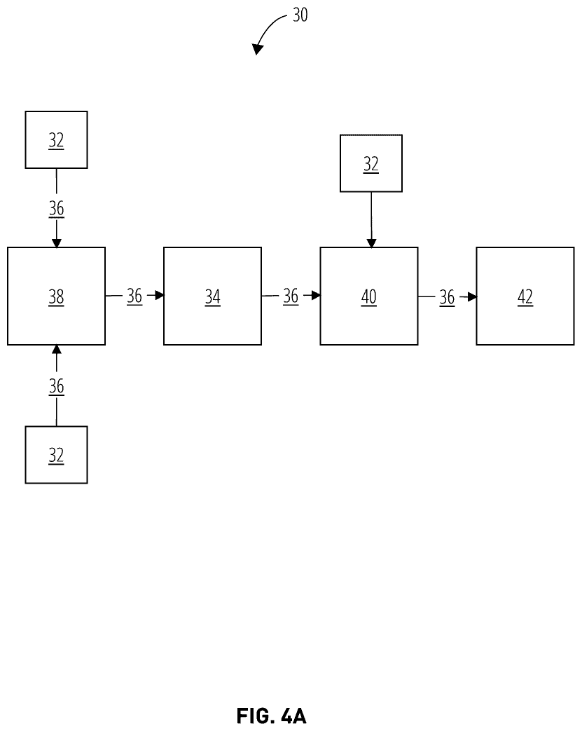

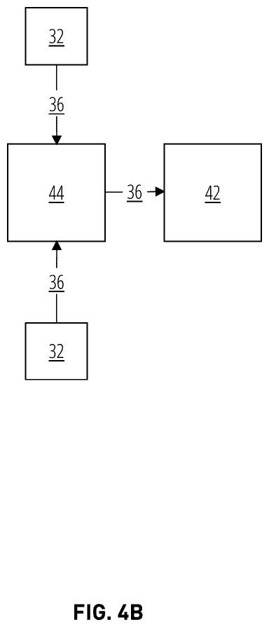

As shown in FIGS. 4A and 4B, the nitration reaction may be conducted in a system that includes one or more microfluidic reactors 30, which include pumps 32 configured to introduce a sulfonate compound solution or a halogenated compound solution and a NR solution to the microfluidic reactor 30. As shown in FIG. 4B, the system may include additional pumps 32, with the additional pumps 32 configured to introduce a solvent, such as DCM, and a base to the microfluidic reactor 30 to conduct the intramolecular condensation reaction. The microfluidic reactor 30 also includes tubing 36, at least one reaction channel 38, 40 (e.g., at least one residence tube), and at least one temperature control system (not shown). As shown in FIG. 4B, the system may also include one or more additional reaction channels 44 in which the intramolecular condensation reaction is conducted. The pumps 32 and reaction channels 38, 40, 44 are in fluid communication with each other by the tubing 36.

The microfluidic reactor 30 is configured to provide control of reagent flow rates, reagent mixing, reaction temperature, and residence time in the reaction channels 38, 40, 44. The components of the microfluidic reactor 30 are configured to be compatible with the reagents, solvents, and other process conditions of the reactions. For example, the components of the microfluidic reactor 30 may be substantially resistant to acidic and basic conditions. The sulfonate compound solution and the NR solution are continuously introduced into, for example, the reaction channel 38 of the microfluidic reactor 30 using the pumps 32, and the nitration reaction occurs in the reaction channel 38 at a desired reaction temperature. The reaction temperature is controlled by the temperature control system.

The microfluidic reactor 30 may, for example, be configured as a glass chip or a glass plate having one or more reaction channels 38, 40, 44 etched in the glass. Internal dimensions of the reaction channels 38, 40, 44 may be between about 1 μm and about 1000 μm. The reaction volume of the microfluidic reactor 30 may depend on the dimensions (e.g., inner diameter, length) of the reaction channels 38, 40, 44 in which the nitration reaction and the intramolecular ring closure reaction are conducted. The reaction channels 38, 40, 44 of the microfluidic reactor 30 have a high surface area to bulk ratio to enable quick and efficient heat dissipation.

The microfluidic reactor 30 may include a back pressure regulator 34 positioned between the reaction channel 38 and the reaction channel 40, in which mixing and or extraction occurs. The back pressure regulator 34 may reduce or eliminate solvent boiling in the microfluidic reactor 30. The second reaction channel 40 may be maintained at room temperature. The emulsion formed in the second reaction channel 40 may be transferred to a separator 42, where the organic (e.g., GLYN/DCM) and aqueous (XNO₃, PTC/MeCN) phases are separately recovered. The separator 42 may be a liquid:liquid phase separator, such as a membrane-based liquid:liquid phase separator. However, other conventional liquid:liquid phase separators may be used. In some embodiments, the separator 42 is a static continuous separator. The nitrate ester may be recovered from the organic solvent (e.g., DCM) by conventional techniques.

The pumps 32 are configured to introduce the sulfonate compound solution and the NR solution into the microfluidic reactor 30 at a desired flow rate, such as at a constant flow rate. Pumps 32 of varying sizes may be used to achieve the desired flow rate and reagent feed ratio. The sodium nitrate and glycidol tosylate are introduced into the microfluidic reactor 30 through inlets (not shown) and combined with mixing in the reaction channel 38. Dichloromethane (DCM) may be pumped into the reaction channel 40 through DCM pump 32. The reaction of the sulfonate compound or halogenated compound and NaNO₃ is based on the flow rate of the reagents through the microfluidic reactor 30 and the length of the first and second reaction channels 38, 40. The flow rate of each of the reagents may be the same or may be different. Alternatively, the desired reagent feed ratio of each of the reagents may be achieved by dilution. Depending on a volume of nitrate ester to be produced, the pumps 32 may be configured as syringe pumps or other conventional pumps.

The tubing 36 is configured to connect the components of the microfluidic reactor to one another, such as to connect the pumps 32 to the reaction channels 38, 40, 44. The tubing 36 may, for example, be resistant to the conditions of the nitration reaction and resistant to the basic conditions of the optional intramolecular condensation reaction. A material of the tubing 36 may, for example, be a polymeric material, a glass material, or a ceramic material. The tubing 36 may be used to transport the nitrate containing compound and the sulfonate compound or the halogenated compound from the pumps 32 to the reaction channel 38. In some embodiments, the tubing 36 is polytetrafluoroethylene (PTFE) or a perfluoroalkoxy alkane (PFA) tubing. The tubing 36 may have an inner diameter of less than or equal to about 1 mm.

The reagents enter the reaction channels 38 and 40 and are combined by mixing, such as by laminar flow mixing, diffusional mixing, etc. The reagents may be combined within a range from milliseconds to seconds due to the small reaction volume of the reaction channels 38, 40.

The reaction channels 38 and 40 are configured to react the reagents (e.g., glycidol tosylate and sodium nitrate) to form the nitrate ester. The reaction channel 44 is configured to react the nitrate ester and byproduct (e.g., DNG) to form additional nitrate ester. The reaction channels 38, 40, 44 are substantially resistant to the reagents and optional solvents used in the reactions. The material of the reaction channels 38, 40, 44 may be resistant to the acidic and basic conditions. The reaction channels 38, 40, 44 may, for example, be formed of a polymeric material, a glass material, or a ceramic material. In some embodiments, the reaction channels 38, 40, 44 are formed from PTFE. In other embodiments, the reaction channels 38, 40, 44 are formed from glass. The reaction channels 38, 40, 44 may have an inner diameter of less than or equal to about 1 mm, such as from about 1 μm to about 1.0 mm (1000 μm), from about 1 μm to about 500 μm, from about 1 μm to about 400 μm, from about 1 μm to about 300 μm, from about 1 μm to about 200 μm, from about 1 μm to about 100 μm, from about 10 μm to about 100 μm, from about 0.1 mm (100 μm) to about 1.0 mm (1000 μm), from about 0.2 mm to about 0.9 mm, from about 0.3 mm to about 0.8 mm, from about 0.4 mm to about 0.8 mm, from about 0.5 mm to about 0.8 mm, from about 0.6 mm to about 0.8 mm, or from about 0.7 mm to about 0.8 mm. A length of the reaction channels 38, 40, 44 may be sufficient for substantially all of the reagents to react within the reaction channels 38, 40, 44 and may be selected depending on the amount of nitrate ester to be produced and the reaction volume of the microfluidic reactor 30. The length of the reaction channels 38, 40, 44 may range from about 30 cm to about 400 cm.

A reaction volume of the microfluidic reactor may be less than or equal to about 40 ml, such as from about 1 μl to about 20 ml, from about 1 μl to about 100 μl, from about 1 μl to about 1000 μl, from about 5 μl to about 100 μl, from about 10 μl to about 90 μl, from about 10 μl to about 80 μl, from about 10 μl to about 50 μl, from about 100 μl to about 1000 μl, from about 1 ml to about 20 ml, from about 5 ml to about 20 ml, from about 5 ml to about 15 ml, from about 5 ml to about 10 ml, from about 10 ml to about 20 ml, from about 15 ml to about 20 ml, or from about 10 ml to about 20 ml.

The temperature control system is configured to maintain the reagents in the reaction channels 38, 40, 44 at a desired temperature during the nitration and intramolecular condensation reactions. The temperature control system may be configured to maintain the reaction channels 38, 40, 44 at a constant temperature during the nitration and intramolecular condensation reactions. The temperature control system is also configured to provide heat to the endothermic nitration reaction and to dissipate heat generated by the exothermic intramolecular condensation reactions. The temperature control system may, for example, be a temperature bath in which the reaction channels 38, 40, 44 and are immersed. The microfluidic reactor 30 has a high surface area to bulk ratio, enabling quick and efficient dissipation or transfer of the heat generated or used by the exothermic and endothermic reactions. A higher degree of temperature control may, therefore, be achieved relative to conventional processes of producing nitrate esters.

The microfluidic reactor 30 may be a commercially available continuous flow reactor, such as CORNING® Low-Flow Advanced-Flow Reactor (LF-AFR), LABTRIX® (reaction volume 1-19.5 μl), PROTRIX® (reaction volume 1-13.5 ml), GRAMFLOW® (reaction volume up to 1 ml), KILOFLOW® (reaction volume 0.8-18 ml), PLANTRIX® (reaction volume 100 ml-4 L), from Chemtrix BV (Echt, the Netherlands). However, the microfluidic reactor 30 may be obtained from other commercial sources.

By producing the nitrate ester in the microfluidic reactor 30, small amounts or small volumes of the nitrate ester, the reagents, or any energetic intermediates are present at a particular time. Therefore, the nitrate ester produced according to embodiments of the disclosure may be produced more safely than conventional processes of producing nitrate esters. However, large amounts of the nitrate ester may be produced according to embodiments of the disclosure since the nitrate ester is produced by a continuous process and since the production of energetic intermediates is minimal. The amount of nitrate ester produced may be easily scaled up to production amounts, such as on the order of kilograms of nitrate ester. Scaleup and throughput may be increased by increasing the reaction path (e.g., the length of the reaction channels 38, 40, 44) or conducting reactions in parallel microfluidic reactors 30. For example, a microfluidic reactor 30 having a 5-ml reaction volume may produce up to about 50 g of nitrate ester per day. A microfluidic reactor 30 having a 200-ml reaction volume may produce production amounts of nitrate ester, such as between about 6 L/hour and about 12 L/hour.

In addition, the use of a microfluidic reactor 30 facilitates emulsion formation and provides consistent temperature throughout the reaction volume, which is important in both endothermic and exothermic reactions. The microfluidic reactor 30 also has the ability to operate through fully remote and automated processes.

While embodiments describe producing GLYN in the microfluidic reactor 30, similar reactors may be used to produce other nitrate esters according to embodiments of the disclosure.

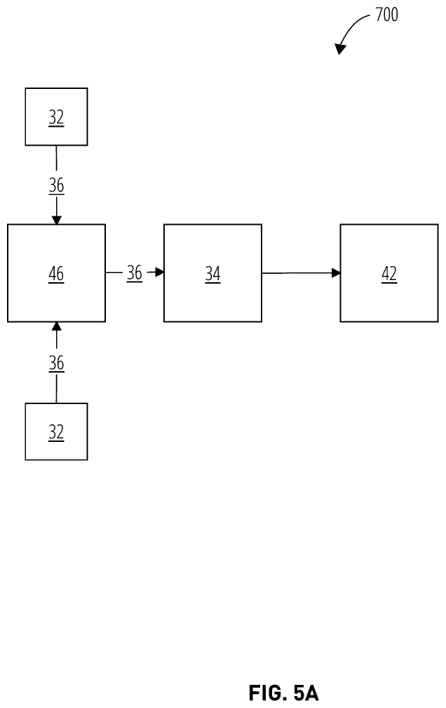

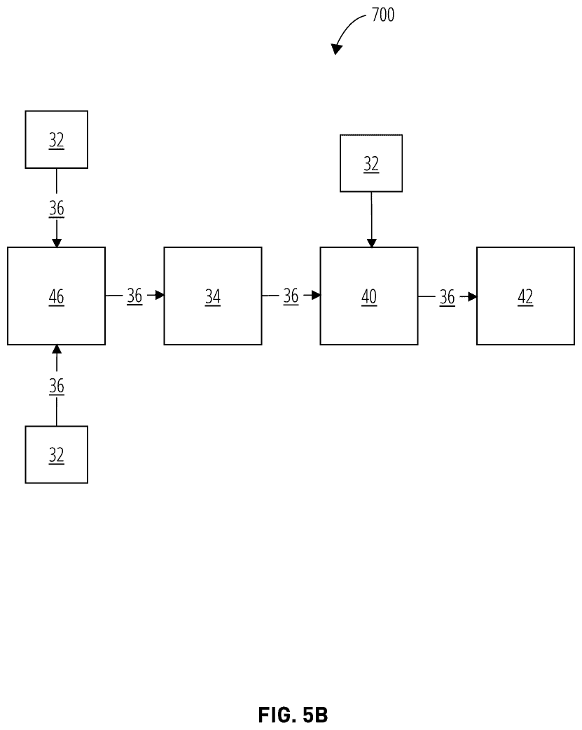

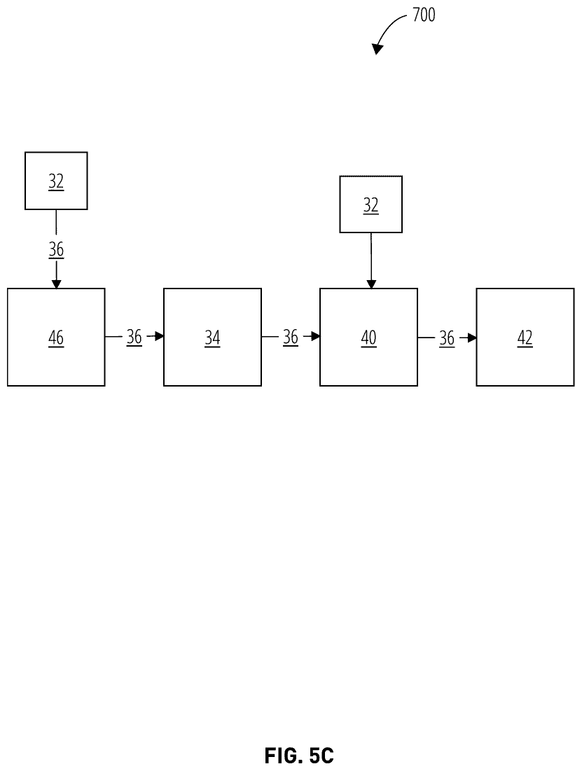

In some embodiments, the nitration reaction and the optional intramolecular condensation reaction (e.g., caustic treatment) may be conducted in a system that includes a plug flow reactor. FIGS. 5A-5C illustrate various embodiments of a plug flow reactor (PFR) system 700. The PFR system 700 includes a PFR 46, pumps 32, a back pressure regulator 34, tubing 36, and a separator 42. The pumps 32, back pressure regulator 34, tubing 36, and separator 42 may be substantially the same as those described with reference to FIGS. 4A and 4B.

As shown in FIG. 5A, the PFR system 700 may include a PFR 46 configured for caustic treatment to convert a byproduct (e.g., DNG) to a nitrate ester (e.g., GLYN). Two pumps 32 are configured to introduce a nitrate ester solution (e.g., a solution containing GLYN and DNG in DCM) and a hydroxide solution into the PFR 46 at desired flow rates, such as at constant flow rates. The pumps 32 may be of varying sizes to achieve the desired flow rate and reagent feed ratio. The caustic treatment in PFR 46 enables the intramolecular ring closure reaction to form additional nitrate ester from byproducts under basic conditions.

The PFR system 700 may, alternatively, include pumps 32 configured to introduce, for example, an alcohol compound solution and a NR solution to the PFR 46, and a pump 32 configured to deliver a DCM solution to the reaction channel 40, as shown in FIG. 5B. The reaction channel 40, which may be a microfluidic reactor channel as described with reference to FIGS. 4A and 4B, is positioned downstream of the PFR 46 and is configured for extraction of the reaction products. The pumps 32 are configured to introduce the solutions at desired flow rates to achieve the intended reagent feed ratios.

As shown in FIG. 5C, the PFR system 700 may, alternatively, include a pump 32 configured to introduce an alcohol compound solution or a halogenated compound solution to the PFR 46, and a pump 32 configured to deliver an organic solvent to the reaction channel 40 for extraction purposes. The halogenated compound solution may include a halogenated compound and an organic catalyst. Although the embodiments illustrated in FIG. 5B and FIG. 5C utilize the reaction channel 40 for extraction purposes, any well-mixed flow system (e.g., continuous stirred-tank reactors, rotating disc contactors, inline static mixers, liquid:liquid extraction columns, etc.) may be used to extract reaction products.

The PFR 46 may include a tube-in-tube plug flow reactor and may be constructed from commercially available components. Plug flow reactors maintain a nearly plug-like flow of reactants along the reactor length with minimal back-mixing, ensuring that each volume element of fluid experiences a similar residence time and reaction conditions. This uniformity may enable more consistent product formation and straightforward scale-up of the nitrate ester. The tube-in-tube configuration may facilitate extended residence times through longer reaction paths compared to microfluidic reactors, while maintaining a compact footprint when coiled. The PFR 46 may be configured to provide control of reagent flow rates and reaction temperature. The components of the PFR 46 are configured to be compatible with the reagents, solvents, and other process conditions of the reactions. For example, the components of the PFR 46 may be substantially resistant to acidic and basic conditions.

The following examples serve to explain embodiments of the disclosure in more detail. These examples are not to be construed as being exhaustive or exclusive as to the scope of this disclosure.

### EXAMPLES

### Example 1

### Microfluidic Reactor

A CORNING® Low-Flow Advanced-Flow Reactor (LF-AFR) in combination with equipment such as low-pulse pumps, flow sensors, liquid-liquid separators, thermostat, etc., were utilized to perform the flow reaction experiments. The microfluidic reactor was similar to the schematic in FIG. 5. Two pumps were connected with tubing to introduce reagents to the microfluidic reactor. The pumps were set to constant flow rates of 1.1 ml/min or 3.0 ml/min. The tubing was chemically resistive PFA tubing and had an inner diameter of 0.063 inch (1.6 mm). The reagents were introduced into the reactor using an LF mixing plate with two inlets to enable mixing of the reagents before entering the residence plates. The tubing containing the reagents was connected to the mixing plate using PFA union joints. The number of residence plates varied, between 2 and 7 plates depending on process conditions. In some conditions, additional residence tubing was used after the reagents left the residence plates to increase residence time, between 30 cm and 412 cm depending on process conditions. Reaction temperature was reported as the target reactor inlet temperature, verified with inline thermocouples placed at various points along the LF-AFR flow path. The measured temperature was typically +/−2° C. of the target. Reagent feed ratios were controlled using set flow rates on pumps. Residence time was controlled by the number of LF mixing or residence plates, residence tube length, and/or the combined flow rate of reagents and was calculated based on the AFR volume held at the reaction temperature and total input flow rate. Actual residence time is approximately 5-10% greater than reported. Back pressure was applied such that no solvent boiling was observed in the reactor. A secondary reactor, an LF-AFR reactor, was used to extract reaction products into an organic solvent, (e.g., DCM). PFA tubing was used to connect the back pressure regulator to an LF mixing plate and additional residence plates as needed. A third pump was used to deliver the DCM to the mixing plate. The mixing and residence plates accomplish thorough mixing of the reaction product mixture of the DCM and extraction of the organic components into the DCM. The final product mixture was separated into organic and aqueous phases using continuous liquid:liquid separation.

### Example 2

### Tosylation and Nitration Reactions

Glycidol tosylate (GT) was synthesized by batch methods and verified to be >95% pure (reagent grade) by NMR analysis. The GT was prepared by dissolving tosyl chloride into an appropriate amount of DCM to bring the neat mixture of glycidol to a 1.0 M concentration. Glycidol and TEA were added to a jacketed reactor and allowed to equilibrate at 0° C. for about 15 minutes. The DCM/tosyl chloride solution was hooked up to a peristaltic pump and added at a rate of 9.99 ml/minute. The addition of the tosyl chloride was completed at approx. 3 hours and the temperature was raised to 20° C. and allowed to react for 4 hours. Amounts of the glycidol, tosyl chloride, and TEA are shown below in Table 1.

TABLE 1
Amounts of glycidol, tosyl chloride, and TEA

| Reagent | Amount | Density | MW | Mmols | eq | Molarity |
|---|---|---|---|---|---|---|
| Glycidol | 132 mLs | 1.11 | 74 | 1980 | 1.0 | 1M |
| Tosyl-Chloride | 376.2 grams | X | 190 | 1980 | 1.0 | x |
| Triethyl Amine (TEA) | 353 mLs | 0.726 | 101 | 2534 | 1.28 | x |

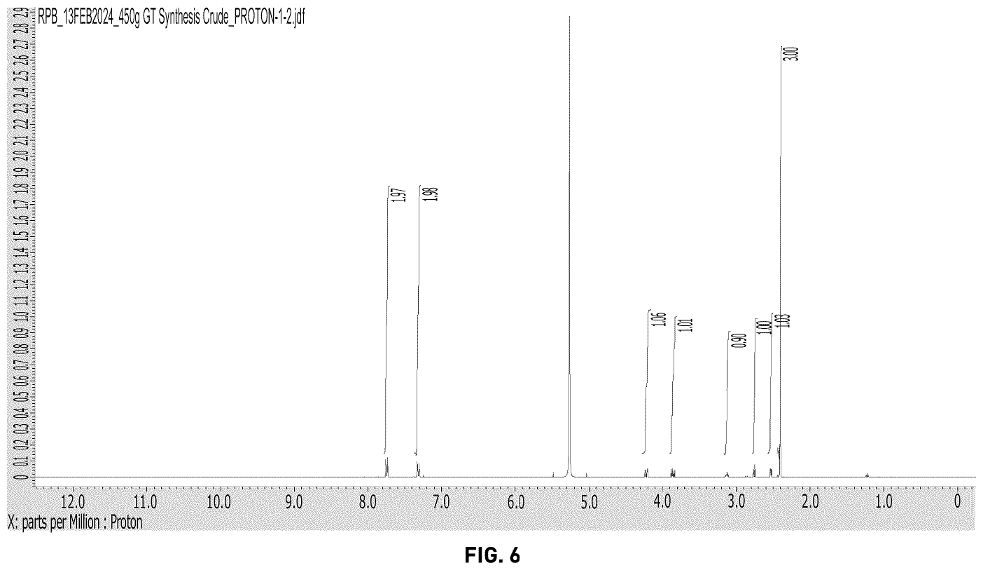

The reaction was filtered through a filter stick into a large round bottom flask, leaving precipitated TEA*HCl in the flask which was washed several times with DCM. The DCM was washed with 1M HCl several times followed by NaHCO₃. The washed filtrate was dried with magnesium sulfate and concentrated using a roto evaporator and Hi-Vac, yielding 429 g GT at about 98% purity. The average yield of the GT was greater than 80% isolated yield. The average purity of the GT was greater than 95% by NMR, as shown in FIG. 6.

The GT solutions were prepared by dissolving GT in reagent grade acetonitrile at varying concentrations. Reagent grade sodium nitrate, polyethylene glycol 1000 (PEG-1000), and distilled water were used to prepare NR solutions. The GT and NR solutions were injected into the reactor described in Example 1. The reaction occurred inside the reactor and the reaction products were collected from the terminal end of the residence tube and analyzed by nuclear magnetic resonance (NMR) spectroscopy and high performance liquid chromatography (HPLC). In particular, the final products were analyzed by sampling an aliquot from the organic phase, drying off the solvent, and recording the weight of the collected material. The collected material was then resolvated in d-chloroform and analyzed by NMR and/or HPLC. The limit of detection for HPLC was 0.01 mg/mL and was based on known standards. Relative molar ratios of all observed products were estimated by NMR, and the total production rate per time was calculated using the total product in the aliquot (per time collected or per volume as appropriated). The reaction products were measured without workup. Table 2 includes the process variables: reactor temperature, GT flow rate, NaNO₃ solution flow rate, GT/NaNO₃ solution, overall flow rate, and residence time.

TABLE 2
Process Variables

| | Reactor | GT Flow | NR Flow | | Overall | Residence |
|---|---|---|---|---|---|---|
| | Temperature | Rate | Rate | | Flow Rate | Time |
| Sample # | (° C.) | (mL/min) | (mL/min) | NR/GT | (mL/min) | (Sec) |
| 1 | 160 | 3.000 | 3.000 | 1 | 6.000 | 30 |
| 2 | 160 | 0.440 | 1.760 | 4 | 2.200 | 81.8 |
| 3 | 160 | 1.100 | 1.100 | 1 | 2.200 | 81.8 |
| 4 | 177.5 | 0.920 | 2.300 | 2.5 | 3.220 | 55.9 |
| 5 | 195 | 1.100 | 1.100 | 1 | 2.200 | 81.8 |
| 6 | 195 | 1.200 | 4.800 | 4 | 6.000 | 30 |
| 7 | 190 | 3.000 | 3.000 | 1 | 6.000 | 30 |

Table 3 shows a results matrix expressed in both total concentration and relative mol % as determined by HPLC analysis using known standards of expected products.

TABLE 3
Results Matrix, product in organic phase by HPLC analysis

| | mg/mL | | | | | | | relative mol % | | | |
|---|---|---|---|---|---|---|---|---|---|---|---|
| | | 1- | 2- | 1,2- | 1,3- | | | | 1,2- | 1,3- | |
| Run | GT | MNG | MNG | DNG | DNG | NG | GLYN | GT | DNG | DNG | GLYN |
| 1 | 4.92 | ND | ND | ND | 0.03 | ND | 0.56 | 81.6% | ND | 0.6% | 17.8% |
| 2 | 3.46 | ND | ND | 0.02 | 0.54 | ND | 2.04 | 42.9% | 0.3% | 8.4% | 48.4% |
| 3 | 3.07 | ND | ND | 0.02 | 0.71 | ND | 1.45 | 45.4% | 0.4% | 13.2% | 41.1% |
| 4 | 1.40 | ND | ND | 0.05 | 2.60 | ND | 3.80 | 11.7% | 0.5% | 27.1% | 60.7% |
| 5 | 0.36 | ND | ND | 0.11 | 2.77 | ND | 3.13 | 3.6% | 1.4% | 34.8% | 60.2% |
| 6 | 0.12 | ND | ND | 0.22 | 5.30 | ND | 3.83 | 0.8% | 1.9% | 46.2% | 51.1% |
| 7 | 2.22 | 0.03 | ND | 0.03 | 1.26 | ND | 3.93 | 19.4% | 0.3% | 13.8% | 66.0% |

The results are expressed as concentration (mg/mL) and relative mol % in the organic phase of: GT=glycidol tosylate, TI=tosylate impurities (includes any unreacted GT), MNG=mononitroglycerine, DNG=dinitroglycerine, NG=trinitroglycerine, GLYN=glycidol nitrate, ND=not detected.

For comparison, Table 4 shows a results matrix expressed in both total concentration and relative mol % as determined by NMR analysis using known standards of expected products.

TABLE 4
Results Matrix, product in organic phase
by NMR analysis (relative mol %)

| | | 1- | 2- | 1,2- | 1,3- | | |
|---|---|---|---|---|---|---|---|
| Run | TI | MNG | MNG | DNG | DNG | NG | GLYN |
| 1 | 93% | ND | ND | ND | ND | ND | 7.0% |
| 2 | 63% | ND | ND | ND | 5.3% | ND | 32% |
| 3 | 67% | ND | ND | ND | 8.8% | ND | 24% |
| 4 | 24% | ND | ND | ND | 25% | ND | 51% |
| 5 | 5.3% | ND | ND | ND | 33% | ND | 61% |
| 6 | ND | ND | ND | ND | 65% | ND | 35% |
| 7 | 33% | ND | ND | ND | 11% | ND | 56% |

As shown in Tables 3 and 4, Run 7 produced the highest % GLYN product at 66% and Run 1 produced the lowest amount at 17.8%. Runs 5 and 6 included the highest percentage of combined GLYN % and DNG %. Thus, two additional experiments were conducted based on the parameters of Runs 5 and 6, and are discussed in Example 3.

Statistical analysis was conducted using JMP software to determine the effects of changing the process variables. The statistical analysis showed the only independent variable to have a significant effect on relative GLYN mol % and production rate was temperature, which showed an increase in values as the temperature increased over the range measured. Other measured variables were inconclusive based on the number of experiments conducted.

### Example 3

### Nitration and Intramolecular Condensation Reactions

The nitration and intramolecular condensation reactions were performed in series in the microfluidic reactor described in Example 1. Glycidol tosylate (GT) was synthesized by batch methods and verified to be >95% pure (reagent grade) by NMR analysis. GT solutions were prepared in reagent grade acetonitrile at varying concentrations. Reagent grade sodium nitrate, polyethylene glycol 1000 (PEG-1000), and distilled water were used to prepare NR solutions. The GT and NR solutions were injected into the reactor described in Example 1. The reaction occurred inside the reactor and the reaction products were collected from the terminal end of the residence tube. The specific process conditions and results for each of the reactions are shown in Tables 5 and 6. Table 5 includes the process variables (reactor temperature, GT flow rate, NR flow rate, NR/GT, overall flow rate, and residence time) that were tested for the nitration reaction.

TABLE 5
Collection run parameters

| | Reactor | GT Flow | NR Flow | | Overall Flow | Residence |
|---|---|---|---|---|---|---|
| Sample | Temperature | Rate | Rate | | Rate | Time |
| Collection# | (° C.) | (mL/min) | (mL/min) | NR/GT | (mL/min) | (Sec) |
| C1 | 195 | 1.100 | 1.100 | 1 | 2.200 | 81.8 |
| C2 | 195 | 1.000 | 4.000 | 4 | 5.000 | 36 |

Table 6 includes the results of the collections by NMR analysis, which are expressed in mole percent (mol %) of the organic phase and by production rate.

TABLE 6
Results of collection runs by NMR analysis

| | Relative mol % | | | Production rate | | |
|---|---|---|---|---|---|---|
| | organic phase | | | (g/hr) | | Total time |
| Sample Name | TI | DNG | GLYN | DNG | GLYN | collected (hrs) |
| C1 | 12% | 23% | 65% | 2.4 | 4.6 | 2.75 |
| C2 | 7.3% | 54% | 39% | 4.1 | 1.9 | 4.5 |

Sample C2, which resulted in 54% DNG and 39% GLYN, was selected for further processing. Thus, after the final collection of sample C2 in DCM, further purification of the product by conversion of the DNG byproduct to GLYN via caustic ring closure using concentrated hydroxide solution (5.5 M) was performed in a flow reactor. The GLYN/DNG was added to the reactor through a first pump and the hydroxide solution was added through a second pump. The phases were separated, in line, into organic (GLYN/DCM) and aqueous.

Table 7 summarizes the run parameters and final results as determined by NMR analysis, with Sample C2f indicating the final collection of the C2 run after caustic treatment and separation into the organic phase, where ND indicates “not detected.” Table 7 includes the process variables (reactor temperature, C2 flow rate, caustic (hydroxide) flow rate, residence time) that were tested and the conversion results, which are expressed in relative mole percent (mol %) of the organic phase.

TABLE 7
Summary of parameters/results of C2 purification by caustic treatment

| | Reactor | C2 Flow | Caustic | Residence | Relative mol % | | |
|---|---|---|---|---|---|---|---|
| Sample | Temperature | Rate | Flow Rate | Time | Organic Phase | | |
| Collection# | (° C.) | (mL/min) | (mL/min) | (Sec) | TI | DNG | GLYN |
| C2f | 74 | 2.000 | 5.000 | 26 | 5.0% | ND | 95% |

Quantitative conversion of DNG to GLYN was observed and the final purity was determined to be 95% (molar) GLYN.

The experiments demonstrated a nucleophilic synthesis route for the formation of nitrate esters using an aqueous system and a phase transfer catalyst. Although the experiments were performed using a flow reactor, batch processes may also be conducted to produce the GLYN. Flow synthesis of GLYN was demonstrated at up to about 8 g/hr and with as little remaining starting material as 5% mol. Longer collection runs of up to about 4.5 hours were also performed, and subsequent purification by caustic ring closure of the DNG side products to GLYN resulted in a final product of up to 95% purity (mol). Continuous collection runs of up to about 8 hours were also conducted for the nitration reaction, as well as collection runs of up to about 18 hours spaced over multiple days.

### Example 4

### Effect of Time on Nitration

Additional collection runs (C3, C4) were performed under the same conditions used in C2 (see Table 5). These runs showed similar results over longer time periods with tosylate impurities <10% (no GT remaining) and GLYN>90% pure after caustic treatment.

Over time it was noticed that PEG-1000 formed an insoluble chelate in the NaNO₃ solution that was prone to clogging in the AFR. To mitigate this issue, a collection was performed with the PEG-1000 dissolved in the GT solution (1 M GT, 20 mM PEG-1000) in acetonitrile rather than the NR solution. This change resulted in a successful collection (C5) under the same reactor conditions as C2-C4 with an overall higher GLYN output.

To mitigate DNG inhibition of GLYN polymerization while preserving reactor life, a plug flow reactor system (PFR) was built with a simple tube-in-tube design using PFA tubing for improved chemical resistance. The PFR system was similar to the schematic in FIG. 5A. The inner tube had an inner diameter of about 1.6 mm (0.625 in), about twice the channel diameter of the AFR, and the length of the PFR was 25 ft. It was assumed to have near ideal plug flow characteristics under these conditions (i.e., good mixing in radial direction, little to no mixing in axial) with an L/D of 480.

A GLYN collection (C6, See Table 8) was performed per the reactor conditions of C2-C4, and caustic treatment (C6f) was performed in the PFR at 90° C. A lower caustic to DNG/GLYN solution ratio was used due to improved kinetics at high temperatures and increased residence time in the PFR. Both the DNG/GLYN solution in DCM and the sodium hydroxide solution were flowed at 2 mL/min for a total flow rate of 4 mL/min and residence time of 113 s (about 5× that possible in the AFR with a lower overall pressure drop). Using the AFR for nitration and the PFR for caustic treatment in series on the same working day resulted in a nearly pure stream of GLYN (see C6f) with no detected DNG, with an average GLYN production of 3.5 g/hr. Results of GLYN collection runs C3-C6 are shown Table 8:

TABLE 8
Summary of GLYN collection runs

| | | | | Total | Total | | | |
|---|---|---|---|---|---|---|---|---|
| | | | Longest | DNG/GLYN | Final | | | |
| Sample | | Total | Single-Shift | Mixture | GLYN | Relative mol % | | |
| Collection | Reactor | hours | Collection | Collected | Collected | Organic Phase | | |
| # | Type | Collected | (hr) | (g) | (g) | GT | DNG | GLYN |
| C3 | AFR | 4.5 | 4.5 | 31.8 | — | ND | 68% | 32% |
| C3f | AFR | 6 | 6 | — | 18.1 | ND | ND | 100% |
| C4 | AFR | 17.8 | 8 | 89.5 | — | ND | 78% | 22% |
| C4f | AFR | 14.75 | 8.5 | — | 64.3 | ND | ND | 100% |
| C5 | AFR | 9.5 | 6 | 38.8 | — | ND | 50% | 50% |
| C5f-1 | AFR | 7.5 | 7.5 | 19.7 | — | ND | 20% | 80% |
| C5f-2 | AFR | 7.5 | 7.5 | — | 14.0 | ND | 5% | 95% |
| C6/C6f | AFR/PFR | 10.5 | 5.5 | — | 36.5 | ND | ND | 100% |

### Example 5

### Nitration in Plug Flow Reactor System

The PFR was selected for further testing of the nitration step (GT to DNG and GLYN). The PFR was coiled into a tight circle to minimize space and wrapped in ceramic insulation to minimize heat loss. For convenience the AFR was used for extraction into DCM, though this may be substituted for any well-mixed flow system. The PFR system was similar to the schematic in FIG. 5B.

Starting solutions were made in the same concentrations as for samples C3-C6 in order to compare AFR and PFR results (i.e., NR solution=5 M NaNO3 in DI water with no pH adjustment, and GT solution=1 M GT+20 mM PEG-1000 in acetonitrile). In this set of experiments, a static continuous separator was used instead of an in-line liquid-liquid separator, as it was discovered that high levels of DNG affected the efficacy of the in-line separators due to cross-solubility in both phases. Back pressure on the PFR was set at 20 bar to prevent excessive solvent boiling. Six total parameter sets were tested in the PFR (replicates not presented here, but showed on trend results), and results are summarized in Tables 9 and 10:

TABLE 9
GT to GLYN in PFR run parameters

| | Reactor | GT | NR | | Overall | |
|---|---|---|---|---|---|---|
| | Temperature | Flow | Flow | | Flow | Residence |
| | Inlet | Rate | Rate | | Rate | Time |
| Run# | (° C.) | (mL/min) | (mL/min) | NR/GT | (mL/min) | (Sec) |
| P-1 | 200 | 1.000 | 4.000 | 4 | 5.000 | 181.2 |
| P-2 | 200 | 2.000 | 4.000 | 2 | 6.000 | 151 |
| P-3 | 186 | 2.000 | 4.000 | 2 | 6.000 | 151 |
| P-4 | 176 | 2.000 | 4.000 | 2 | 6.000 | 151 |
| P-5 | 166 | 2.000 | 4.000 | 2 | 6.000 | 151 |
| P-6 | 171 | 2.000 | 4.000 | 2 | 6.000 | 151 |

TABLE 10
Results GT to GLYN in PFR runs by NMR analysis

| Sample | Relative mol % organic phase | | | Production rate (g/hr) | |
|---|---|---|---|---|---|
| Name | TI | DNG | GLYN | DNG | GLYN |
| P-1 | 2.4% | 92.3% | 5.3% | 1.5 | 0.1 |
| P-2 | 2.8% | 87.3% | 9.9% | 1.6 | 0.1 |
| P-3 | 2.2% | 79.1% | 18.8% | 4.9 | 0.8 |
| P-4 | 4.4% | 44.4% | 51.2% | 3.6 | 2.7 |
| P-5 | 4.4% | 41.4% | 54.2% | 4.2 | 3.6 |
| P-6 | 2.8% | 47.9% | 49.3% | 4.6 | 3.1 |

In the first PFR run (P-1), flow and temperature were similar to the AFR run in C2 (see Table 5), only varying the total residence time due to the larger residence volume. In this case residence time was increased by five times compared to C2, and it was found that the inlet temperature could be held higher (200° C. in PFR instead of 195° C. in AFR) due to lower heat loss in the PFR system. In this case, the GT was nearly fully converted to DNG at >92%, but had a lower than expected output rate, which, without being bound by theory, was likely due to decomposition into water soluble products. No GT was detected, but tosylate impurities were seen in NMR analysis. In P-2 a lower NR/GT ratio and residence time were tested and showed similar overall production but improved DNG and GLYN ratios compared to P-1. In P-3 through P-6, the temperature was varied while keeping the other parameters constant to test how low the temperature could be set before seeing appreciable tosylate impurities (including GT). When the inlet temperature dropped below 170° C., GT could be seen in the NMR analysis and tosylate impurities in total reached just above 4%. A performing temperature for a NR/GT of 2 and overall residence time of 151 s in the PFR was found to be ˜170° C. to maximize the total output of DNG and GLYN while minimizing GT and TI. The temperature difference from inlet to outlet of the PFR was on average about 3° C. compared to an average of about 10° C. in the AFR.

The collection of GLYN/DNG in DCM solutions from runs P2-P6 were caustic treated in a second PFR of the same specifications under conditions similar to run C6f, resulting in no detectable DNG and >98% GLYN with <2% tosylated impurities by NMR analysis. The combined total output of both nitration and caustic ring closure in separate PFR systems was about 3 g GLYN/hr.

### Example 6

### Nitration of 1,3-Dichloropropanol

Batch experimentations were performed to confirm reactivity of phase transfer catalyzed nucleophilic substitution of solubilized sodium nitrate with other leaving groups (i.e., leaving groups other than tosylates and mesylates). Previously this methodology was demonstrated on leaving groups such as tosylates (see Examples 1-4). 1,3 dichloropropanol (1,3-DCP) was used to test batch performance with less reactive leaving groups (e.g., halogens). The first series of reaction parameters were designed to be similar to the previously established tosylate replacement parameters (see Examples 1-4). Successful nitration was observed in standard reaction conditions (1,3-DCP, PEG-1000, nitrating reagent, and acetonitrile heated to 80° C. for 24 hrs), reactions without acetonitrile, and reactions without acetonitrile and PEG-1000. No nitration was observed in reactions at room temperature and reactions at room temperature without acetonitrile.

After performing batch experimentations, a homogenous system (i.e., no PTC) was developed using an organic catalyst (i.e., DABCO) to enhance the substitution reaction. Using 5M NaNO₃ as a reaction medium and an NMR reference peak of AB-quartet of 1,3-dinitro propanol at ˜4.7 ppm, reagents were reacted at 85° C. for 24 hours unless otherwise noted, as shown in Table 11:

TABLE 11
Reactions of DABCO in 5M aqueous sodium nitrate
for the nucleophilic substitution of 1,3-DCP

| Reaction | Conditions | Nitration Observed By NMR |
|---|---|---|
| 1 | 5M NaNO₃ | Yes |
| | 10 mol % DABCO | |
| | 85° C. | |
| 2 | 5M NaNO₃ | Yes |
| | 25 mol % DABCO | |
| | 85° C. | |
| 3 | 5M NaNO₃ | Yes |
| | 50 mol % DABCO | |
| | 85° C. | |
| 4 | 5M NaNO₃ | No |
| | 50 mol % DABCO | |
| | Room temp | |
| 5 | 5M NaNO₃ | No |
| | 50 mol % DABCO | |
| | Room temp | |
| | No pH control | |

Reactions 1-3 yielded observable nitration indicating the success of the monophasic nucleophilic substitution of NO₃ with Cl using a tertiary amine as a catalyst in a monophasic environment.

### Example 7

### Nitration of 1,3-Dichloropropanol in a PFR System

Nitration of 1,3-DCP without the use of a PTC was demonstrated in a 25 ft PFR system. The PFR system was similar to the schematic in FIG. 5C. Solutions of 1,3-DCP, sodium nitrate, DABCO catalyst and sodium hydroxide for pH adjustment in DI water were pre-mixed as a reaction solution (hereafter referred to as R-Sol). Stability of these reaction solutions were tested at room temperature over multiple days with no detectable reaction taking place, as observed in the small-scale batch tests (see Example 6).

Back pressure (between 5-11 bar in all reactions) was added as needed to prevent boiling. The reaction conditions used are shown in Table 12, and reaction results are shown in Table 13:

TABLE 12
1,3-DCP Nitration in PFR Conditions

| | Reactor | Flow Rate R- | Residence |
|---|---|---|---|
| Run# | Temperature (° C.) | Sol (mL/min) | Time (Sec) |
| D-1 | 150 | 5.000 | 181 |
| D-2 | 150 | 1.000 | 905 |
| D-3 | 170 | 1.667 | 543 |
| D-4 | 190 | 5.000 | 181 |
| D-5 | 190 | 1.000 | 905 |

TABLE 13
Results 1,3-DCP Nitration in PFR runs by NMR analysis

| Sample | Relative mol % organic phase | | | Production rate (g/hr) | |
|---|---|---|---|---|---|
| Name | 1,3-DCP | 1,3-DNP | GLYN | 1,3-DNP | GLYN |
| D-1 | 26.0% | 41.8% | 43.5% | 0.18 | 0.1 |
| D-2 | 35.0% | 65.0% | ND | 0.08 | ND |
| D-3 | 24.9% | 75.1% | ND | 0.21 | ND |
| D-4 | 31.0% | 69.0% | ND | 0.26 | ND |
| D-5 | 65.9% | 34.1% | ND | 0.10 | ND |

Nitration was observed for all conditions studied, demonstrating success of the reaction in flow. In D-1, the 1,3-DCP reactant, 1,3-DNP and GLYN were observed in the organic phase collected. Without being bound by theory, the presence of GLYN was likely due to the high pH of the solution, which caused some caustic ring closure during the course of the reaction. Due to the high solubility of 1,3-DCP in water, it was expected that the majority would not be observable in the water layer. Therefore, in this case, the overall production of 1,3-DNP or GLYN was seen as the most important metric. Additional experiments in the PFR (D-2, D-4, and D-5) were performed at higher initial 1,3-DCP concentrations in the reactant solution and varying levels of DABCO catalyst and sodium hydroxide with similar results.

While the disclosure is susceptible to various modifications and alternative forms, specific embodiments have been shown by way of example in the drawings and have been described in detail herein. However, the disclosure is not intended to be limited to the particular forms disclosed. Rather, the disclosure encompasses all modifications, equivalents, and alternatives falling within the scope of the disclosure as defined by the following appended claims and their legal equivalents.

What is claimed is:

1. A method of producing a nitrate ester, comprising:
reacting an alcohol compound and an electrophilic compound to form a sulfonate compound; and
reacting the sulfonate compound with a nitrate containing compound to form the nitrate ester.

2. The method of claim 1, wherein reacting an alcohol compound and an electrophilic compound comprises reacting glycidol, 1,3-dichloropropanol, isosorbide, ethylene glycol, propylene glycol, glycerol, 1,4-butanediol, pentaerythritol, or erythritol and the electrophilic compound to form the sulfonate compound.

3. The method of claim 1, wherein reacting an alcohol compound and an electrophilic compound comprises reacting the alcohol compound with tosyl chloride, mesyl chloride, mesyl iodide, or tosyl iodide to form the sulfonate compound.

4. The method of claim 1, wherein reacting the sulfonate compound with a nitrate containing compound comprises reacting the sulfonate compound with the nitrate containing compound in the presence of a phase transfer catalyst to form the nitrate ester.

5. The method of claim 4, wherein reacting the sulfonate compound with the nitrate containing compound in the presence of a phase transfer catalyst comprises reacting the sulfonate compound with the nitrate containing compound in the presence of polyethylene glycol.

6. The method of claim 1, wherein reacting an alcohol compound and an electrophilic compound comprises reacting glycidol with tosyl chloride to form glycidol tosylate.

7. The method of claim 1, wherein reacting the sulfonate compound with a nitrate containing compound comprises reacting the sulfonate compound with the nitrate containing compound in the presence of a phase transfer catalyst comprising a linear polyether, a cyclic polyether, a substituted linear polyether, a substituted cyclic polyether, or a combination thereof to form the nitrate ester.

8. The method of claim 1, wherein reacting the sulfonate compound with a nitrate containing compound comprises reacting the sulfonate compound with sodium nitrate, calcium nitrate, potassium nitrate, or lithium nitrate to form the nitrate ester.

9. The method of claim 1, wherein reacting an alcohol compound and an electrophilic compound to form a sulfonate compound and reacting the sulfonate compound with a nitrate containing compound comprises reacting the alcohol compound and the electrophilic compound to form the sulfonate compound and reacting the sulfonate compound with the nitrate containing compound in a microfluidic reactor or in a plug flow reactor.

10. A method of producing glycidol nitrate, comprising:
combining a solution comprising glycidol sulfonate and an organic solvent with an aqueous solution comprising a metal nitrate compound and a phase transfer catalyst to form an emulsion;
reacting the glycidol sulfonate with the metal nitrate compound to form glycidol nitrate;
transferring the glycidol nitrate to the organic solvent and metal ions and nitrate ions to the aqueous solution; and
recovering the glycidol nitrate from the organic solvent.

11. The method of claim 10, wherein combining a solution comprising glycidol sulfonate and an organic solvent with an aqueous solution comprising a metal nitrate compound and a phase transfer catalyst comprises combining the solution comprising glycidol sulfonate and acetonitrile with the aqueous solution.

12. The method of claim 10, wherein reacting the glycidol sulfonate with the metal nitrate compound to form glycidol nitrate comprises forming glycidol nitrate and dinitroglycerol in a microfluidic reactor or a plug flow reactor.

13. The method of claim 12, further comprising reacting the dinitroglycerol with a base to form additional glycidol nitrate.

14. The method of claim 13, further comprising separating the additional glycidol nitrate from the dinitroglycerol.

15. A method of producing a nitrate ester, comprising:
reacting a halogenated compound with a nitrate containing compound to form a nitrate ester.

16. The method of claim 15, wherein reacting a halogenated compound with a nitrate containing compound comprises:
reacting the halogenated compound with an organic catalyst to form an activated complex of the halogenated compound; and
reacting the activated complex of the halogenated compound with the nitrate containing compound to form the nitrate ester.

17. The method of claim 16, wherein reacting the halogenated compound with an organic catalyst comprises reacting the halogenated compound with a tertiary amine compound to form the activated complex of the halogenated compound.

18. The method of claim 16, wherein reacting the halogenated compound with an organic catalyst comprises reacting the halogenated compound with one or more of 1,4-diazabicyclo[2.2.2]octane (DABCO), triethylamine (TEA), N,N-dimethylaminopyridine (DMAP), 1,8-diazabicyclo[5.4.0]undec-7-ene (DBU), N-methylmorpholine (NMM), 1,4-diazabicyclo[2.2.1]heptane (DABCH), N,N-diisopropylethylamine (DIPEA), quinuclidine, N-methylpiperidine, N,N,N′,N′-tetramethylethylenediamine (TMEDA), 1,5-diazabicyclo[4.3.0]non-5-ene (DBN), and 2,2,6,6-tetramethylpiperidine (TMP) to form the activated complex of the halogenated compound.

19. The method of claim 16, wherein reacting the halogenated compound with an organic catalyst comprises reacting 1,3-dichloropropanol with DABCO.

20. The method of claim 15, wherein reacting a halogenated compound with a nitrate containing compound comprises maintaining the halogenated compound and the nitrate containing compound at a temperature within a range of from about 60° C. to about 200° C. to form the nitrate ester.
